(19) United States
(12) Patent Application Publication
(10) Pub. No.: US 2025/0194491 A1
(43) Pub. Date: Jun. 19, 2025

(54) Wheat Variety PMWH149312414

(71) Applicant: BASF SE, Ludwigshafen am Rhein (DE)

(72) Inventors: David G Bonnett, Sabin, MN (US); Danielle M. Fiebelkorn Wrucke, Sabin, MN (US)

(73) Assignee: BASF SE, Ludwigshafen am Rhein (DE)

(21) Appl. No.: 18/980,168

(22) Filed: Dec. 13, 2024

Related U.S. Application Data
(60) Provisional application No. 63/611,856, filed on Dec. 19, 2023.

(51) Int. Cl. A01H 6/46; A01H 5/10

(52) U.S. Cl. CPC A01H 6/4678; A01H 5/10

(57) ABSTRACT

A wheat variety designated PMWH149312414, the plants and seeds of wheat variety PMWH149312414, methods for producing a wheat plant produced by crossing the variety PMWH149312414 with another wheat plant, and hybrid wheat seeds and plants produced by crossing the variety PMWH149312414 with another wheat line or plant, and the creation of variants by backcrossing, mutagenesis or transformation of variety PMWH149312414 are disclosed. Methods for producing other wheat varieties or breeding lines derived from wheat variety PMWH149312414 and to wheat varieties or breeding lines produced by those methods are also provided.

### FIELD OF THE DISCLOSURE

This disclosure relates to wheat (Triticum aestivum L.) breeding, specifically relating to a new and distinctive wheat variety designated PMWH149312414.

### BACKGROUND OF THE DISCLOSURE

There are numerous steps involving significant intervention in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These traits may include, but are not limited to, higher seed yield, resistance to diseases and/or insects, better stems and roots, tolerance to drought and/or heat, resistance to herbicides, altered milling properties, abiotic stress tolerance, improvements in compositional traits, and better agronomic characteristics.

Wheat is grown worldwide and is the most widely adapted cereal. Wheat may be classified into six different market classes. Five of these, including common wheat, hard red winter, hard red spring, soft red winter, and white, belong to the species Triticum aestivum L., and the sixth, durum, belongs to the species Triticum turgidum L. Common wheats are used in a variety of food products, including but not limited to grain, flour, baked goods, cereals, pasta, bread, cookies, cakes, crackers, and noodles. In general, the hard wheat classes are milled into flour used for breads and the soft wheat classes are milled into flour used for pastries and crackers. It may also be used to produce beverages, livestock feed, biofuel, straw, construction materials and starches. Wheat starch is also used in the paper industries, as laundry starches, and in other products.

### SUMMARY OF VARIOUS ASPECTS OF THE DISCLOSURE

Seeds of the wheat variety PMWH149312414 are provided. Also provided are plants produced by growing the seed of the wheat variety PMWH149312414, as well as the derivatives of such plants. Further provided are plant parts, including cells, plant protoplasts, plant cells of a tissue culture from which wheat plants can be regenerated, plant calli, plant clumps, and plant cells that are intact in plants or parts of plants, such as leaves, stems, roots, root tips, anthers, pistils, seed, grain, pericarp, embryo, pollen, ovules, scutellum, hypocotyl, spike, floret, awn, lemma, shoot, tissue, petiole, cells, and meristematic cells, and the like.

In a further aspect, a composition comprising a seed of wheat variety PMWH149312414 comprised in plant seed growth media is provided. The plant seed growth media can be, for example, a soil or synthetic cultivation medium. The growth medium may be comprised in a container or may, for example, be soil in a field. Plant seed growth media are well known to those of skill in the art and include, but are in no way limited to, soil or synthetic cultivation medium. Advantageously, plant seed growth media can provide adequate physical support for seeds and can retain moisture and/or nutritional components. Examples of characteristics for soils that may be desirable in certain aspects can be found, for instance, in U.S. Pat. Nos. 3,932,166 and 4,707,176. Synthetic plant cultivation media include those known in the art and may, for example, comprise polymers or hydrogels. Examples of such compositions are described in U.S. Pat. No. 4,241,537.

A tissue culture of regenerable cells of the wheat variety PMWH149312414 is provided, as well as plants and plant parts regenerated therefrom, wherein the regenerated wheat plant is capable of expressing all the physiological and morphological characteristics of a plant grown from the wheat seed designated PMWH149312414.

A wheat plant comprising a locus conversion or single locus conversion of the wheat variety PMWH149312414, wherein the wheat plant is otherwise capable of expressing all (except for those conferred/changed by the locus conversion) the physiological and morphological, or phenotypic, characteristics of the wheat variety PMWH149312414 is provided. The locus conversion may be introduced by backcrossing or by genetic transformation, and may comprise, for example, a transgenic, intragenic, cisgenic, targeted genome-edited, translocated, deleted, duplicated, inverted, recombined or mutated gene or locus which has been introduced by transformation, targeted genome editing, (chemical or radiation-induced) mutagenesis, translocation or by chromosome rearrangement (including but not limited to chemical or radiation-induced translocation or chromosome rearrangement) into the wheat variety PMWH149312414 or a progenitor thereof. “Genetic transformation”, as used herein, refers to plant transformation, targeted genome editing, mutagenesis (such as chemical or radiation-induced mutagenesis, like EMS mutagenesis), translocation or chromosome rearrangement (including but not limited to chemical or radiation-induced translocation or chromosome rearrangement), wherein at least one change/modification is made to the plant genome (including a substitution of one or some nucleotides in other nucleotides, a deletion of one or more nucleotides, an addition of one or more nucleotides, the same nucleotide sequence at another position in the genome, the same nucleotide sequence in the opposite orientation in the genome (inversion), a duplication of one or more nucleotides, etc.). The locus conversion may, for example, comprise a dominant or recessive allele or a genetic modification introduced by manipulation of the plant genome or a genome-edited, translocated, deleted, duplicated, inverted, recombined or mutated gene, including but not limited to a gene or locus introduced or modified by introducing a transgene by transformation, targeted genome editing, mutagenesis, translocation or by chromosome rearrangement. One method to identify mutations is targeting induced local lesions in genomes (“TILLING”), that combines mutagenesis (e.g., using a chemical mutagen such as ethyl methanesulfonate) with a sensitive DNA screening-technique that identifies mutations in a target gene. The locus conversion may confer potentially any trait upon the converted plant, including, but not limited to, herbicide resistance, insect resistance, resistance to bacterial, fungal, or viral disease, male fertility (such as a fertility restoration trait/gene to restore a male sterility trait/gene) or sterility (such as male sterility (like cytoplasmic or genic male sterility)), improved pollination or pollen receptivity, improved female receptivity, abiotic stress, altered phosphorus content, altered antioxidants, altered essential amino acids, and altered nutritional quality, such as altered starch, sugars, non-digestible carbohydrate, protein, oil or fatty acids. The altered trait can be compared to a wheat variety PMWH149312414 not comprising the locus conversion.

Wheat plants are provided which comprise a transgene, mutation or targeted genome edited modification and which were produced by genetic transformation, such as transforming, mutating or genome editing the plant, plant part, seed or cell of wheat variety PMWH149312414, or which had the transgene, mutation or the genome-edited modification introgressed through back-crossing.

Methods for producing a wheat plant are provided in which plant breeding techniques are applied to a wheat plant grown from seed of wheat variety PMWH149312414 comprising a locus conversion, or to a plant grown from seed of a cross of such a wheat plant to a different wheat plant.

First generation (F₁) hybrid wheat seed produced by crossing a plant of the wheat variety PMWH149312414 to a second wheat plant are provided. Also provided are the F₁ hybrid wheat plants grown from the hybrid seed produced by crossing the wheat variety PMWH149312414 to a second wheat plant. Still further provided are the seeds of an F₁ hybrid plant produced with the wheat variety PMWH149312414 as one parent, the second generation (F₂) hybrid wheat plant grown from the seed of the F₁ hybrid plant, and the seeds of the F₂ hybrid plant.

Methods of producing wheat seeds are provided which comprise crossing a plant of the wheat variety PMWH149312414 to any second wheat plant, including itself or another plant of the variety PMWH149312414. For example, the method of crossing can comprise the steps of: (a) planting seeds of the wheat variety PMWH149312414; (b) cultivating wheat plants resulting from said seeds until said plants bear flowers; (c) allowing fertilization of the flowers of said plants by its own pollen or by pollen from adjacent other wheat plants, or allowing fertilization of flowers from adjacent other wheat plants by pollen of said plants; and (d) harvesting selfed or F₁ seeds produced from said plants, or produced on said pollinated other adjacent wheat plants.

A method of producing hybrid wheat seeds is provided which comprises crossing the wheat variety PMWH149312414 to a second, distinct wheat plant that is nonisogenic to the wheat variety PMWH149312414. For example, the crossing can comprise the steps of: (a) planting seeds of wheat variety PMWH149312414 and a second, distinct wheat plant, (b) cultivating the wheat plants grown from the seeds until the plants bear flowers; (c) cross pollinating a flower on one of the two plants with the pollen of the other plant, and (d) harvesting the seeds resulting from the cross pollinating.

A method for developing a wheat plant in a wheat breeding program is provided comprising: (a) obtaining or providing a wheat plant, or its parts, of the variety PMWH149312414; and (b) employing said plant or parts as a source of breeding material in a plant breeding program such as using plant breeding techniques. In the method, the plant breeding techniques may be selected, for example, from recurrent selection, mass selection, selfing, bulk selection, backcrossing, pedigree breeding, genetic marker-assisted selection, mutation, introduction of a transgene by transformation and (site-directed) genome modification. The wheat plant of variety PMWH149312414 may be used as the male or female parent.

A method of producing a wheat plant derived from the wheat variety PMWH149312414 is provided, the method comprising the steps of: (a) preparing a progeny plant derived from wheat variety PMWH149312414 by crossing a plant of the wheat variety PMWH149312414 with a second wheat plant; and (b) crossing the progeny plant with itself or a second plant to produce a progeny plant of a subsequent generation which is derived from a plant of the wheat variety PMWH149312414. Optionally, the method may further comprise: (c) crossing the progeny plant of a subsequent generation with itself or a second plant; and (d) repeating steps (b) and (c) for at least, for example 2, 3, 4, 5, 6 or more additional generations to produce an inbred or hybrid wheat plant derived from the wheat variety PMWH149312414. Also provided is a plant produced by this and other methods described herein.

A method of producing a wheat plant derived from the wheat variety PMWH149312414 can, for example, further comprise: (a) crossing the wheat variety PMWH149312414-derived wheat plant with itself or another wheat plant to yield additional wheat variety PMWH149312414-derived progeny wheat seed; (b) growing the progeny wheat seed of step (a) under plant growth conditions to yield additional wheat variety PMWH149312414-derived wheat plants; and (c) repeating the crossing and growing steps of (a) and (b) to generate further wheat variety PMWH149312414-derived wheat plants. Steps (a) and (b) can be repeated if desired at least 1, 2, 3, 4, 5, 6 or more times. Also provided is a wheat plant produced by this and other methods described herein.

The production of doubled haploids can also be used for the development of plants with a homozygous phenotype in the breeding program. Methods for producing doubled haploid wheat plants from wheat variety PMWH149312414 are provided. For example, a wheat plant produced by growing a seed of the cross of wheat variety PMWH149312414 with a different wheat plant or plant part can be crossed with another plant to form haploid cells. The chromosomes of the haploid cells can be doubled according to established processes to form doubled haploid cells which are grown into a doubled haploid wheat plant or plant part. Haploid seed generated from a cross of a wheat plant disclosed herein with a different wheat plant can be doubled to produce a wheat plant having doubled haploid chromosomes.

Methods for cleaning, conditioning, or applying a seed treatment to the seed of wheat variety PMWH149312414 are provided.

Methods of milling the seed of wheat variety PMWH149312414 and the flour produced from such milling are provided. The flour may include a cell of wheat variety PMWH149312414.

### DETAILED DESCRIPTION OF VARIOUS ASPECTS OF THE DISCLOSURE

The present disclosure relates to a new and distinctive wheat (Triticum aestivum L.) variety designated PMWH149312414, its seeds, plants, plant parts and hybrids thereof. Wheat variety PMWH149312414 is a common hard white spring type wheat bred for spring wheat growing regions of the United States and for parts of Canada. One of the potential uses of the wheat variety PMWH149312414 will be for production of hybrid seed.

Also provided are methods for making PMWH149312414 that comprise crossing wheat variety PMWH149312414 with another wheat plant and processes for making a wheat plant containing in its genetic material one or more traits introgressed into PMWH149312414 through backcross conversion and/or transformation or genome modification, and to the wheat seed, plant and plant parts produced thereby. Variants of wheat PMWH149312414 created by transformation or genome modification, or mutagenesis, as well as a hybrid wheat seed, plant or plant part produced by crossing the variety PMWH149312414 or a locus conversion of PMWH149312414 with another wheat variety are also provided.

Wheat variety PMWH149312414 has shown uniformity and stability for all traits, as described in the variety description information provided herein. It has been self-pollinated a sufficient number of generations, with careful attention to uniformity of plant type to ensure homozygosity and phenotypic stability. The line has been increased with continued observation for uniformity. No variant traits have been observed or are expected in PMWH149312414, other than as described, for example, in the variety description information below.

Field crops are bred through techniques that take advantage of the plant's method of pollination, such as self-pollination, sib-pollination or cross-pollination. As used herein, the term cross-pollination includes pollination with pollen from a flower on a different plant from a different family or line and does not include self-pollination or sib-pollination. Wheat plants (Triticum aestivum L.) are recognized to be naturally self-pollinated plants which, while capable of undergoing cross-pollination, rarely do so in nature. Thus, intervention for control of pollination is needed for the establishment of superior varieties.

In another aspect, the disclosure is directed to methods for producing a wheat plant by crossing a first parent wheat plant with a second parent wheat plant, wherein the first or second wheat plant is the wheat plant from the variety PMWH149312414. In an aspect, the first and second parent wheat plants may be from the variety PMWH149312414 (i.e., self-pollination). Any methods using the variety PMWH149312414 are part of this disclosure: selfing, backcrosses, hybrid breeding, and crosses to populations. Any plants produced using variety PMWH149312414 as a parent are within the scope of this disclosure. In certain aspects, the disclosure is also directed to cells that, upon growth and differentiation, produce a variety having essentially all of the morphological and physiological characteristics of PMWH149312414, except for the morphological or physiological characteristics changed by a locus conversion or by any added or deleted loci/traits that changes some morphological or physiological characteristic(s) (such as a change in herbicide tolerance, (viral or fungal) disease resistance, insect resistance, plant height, maturity date, seed color, seed size, seed weight, seed number, male sterility, restoration genes for male sterility that restore male fertility). The present disclosure additionally contemplates, in various aspects, a wheat plant regenerated from a tissue culture of variety PMWH149312414.

Provided are methods of producing progeny with a new combination of genetic traits by cross pollinating one wheat plant with another by emasculating flowers of a designated female plant and pollinating the female parent with pollen from the designated male parent. Suitable methods of cross-pollination of wheat plants are described, for example, in U.S. Pat. No. 8,809,654, which is herein incorporated by reference, but other methods can be used, or modified, as is known to those skilled in the art.

A cross between two different homozygous lines produces a uniform population of hybrid plants that may be heterozygous for many gene loci. A cross of two heterozygous plants each that differ at a number of gene loci will produce a population of plants that differ genetically and will not be uniform. Regardless of parentage, plants that have been self-pollinated and selected for type for many generations become homozygous at almost all gene loci and produce a uniform population of true breeding progeny. The term “homozygous plant” is hereby defined as a plant with homozygous genes at 95% or more of its loci.

Choice of breeding or selection methods depends on the mode of plant reproduction, the heritability of the trait(s) being improved, and the type of variety used commercially (e.g., F₁ hybrid variety, pureline variety, etc.). For highly heritable traits, a choice of superior individual plants evaluated at a single location will be effective, whereas for traits with low heritability, selection can be based on mean values obtained from replicated evaluations of families of related plants. Popular selection methods which can be used include pedigree selection, modified pedigree selection, mass selection, and recurrent selection.

The complexity of inheritance influences choice of the breeding method. For example, pedigree breeding, backcross breeding, single seed descent, and bulk breeding, which are each described in U.S. Pat. No. 8,809,654 (incorporated herein by reference), can be used. Each wheat breeding program may include a periodic, objective evaluation of the efficiency of the breeding procedure. Evaluation criteria vary depending on the goal and objectives, but may include gain from selection per year based on comparisons to an appropriate standard, overall value of the advanced breeding lines, and number of successful varieties produced per unit of input (e.g., per year, per dollar expended, etc.).

Various recurrent selection techniques can be used to improve quantitatively inherited traits controlled by numerous genes. The use of recurrent selection in self-pollinating crops depends on the ease of pollination and the number of hybrid offspring from each successful cross. Recurrent selection can be used to improve populations of either self- or cross-pollinated crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued. Plants from the populations can be selected and selfed to create new varieties.

Wheat variety PMWH149312414 can be used as the female or the male parent in biparental crosses in order to develop new and valuable wheat varieties or hybrids. Wheat normally self-pollinates in nature. Cross pollination of one wheat plant with another to produce progeny with a new combination of genetic traits, can be carried out according to methods known to those skilled in the art. Wheat cross-pollination is achieved by emasculating flowers of a designated female plant and pollinating the female parent with pollen from the designated male parent. Methods of cross-pollinating wheat plants for use in selection and advancement are described, for example in U.S. Pat. No. 9,282,712, the disclosure of which is incorporated herein by reference in its entirety.

Plant breeding methods may include analysis, comparison and characterization of the plant genome and the use of molecular markers, including techniques such as Starch Gel Electrophoresis, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), Single Nucleotide Polymorphisms (SNPs) and Quantitative Trait Loci (QTL) mapping.

Molecular markers can also be used during the breeding process for the selection of qualitative traits. For example, markers closely linked to alleles or markers containing sequences within the actual alleles of interest can be used to select plants that contain the alleles of interest during a crossing or backcrossing breeding program. The markers can also be used to select for the genome of the recurrent parent and against the markers of the donor parent. Using this procedure one can minimize the amount of genome from the donor parent that remains in the selected plants. It can also be used to reduce the number of crosses back to the recurrent parent needed in a backcrossing program.

The production of doubled haploids can also be used for the development of homozygous lines in the breeding program and in the production of, for example, hybrid wheat using variety PMWH149312414. Doubled haploids are produced by the doubling of a set of chromosomes (IN) from a heterozygous plant to produce a completely homozygous individual. This can be advantageous because the process omits the generations of selfing needed to obtain a homozygous plant from a heterozygous source. Hybrid wheat can be produced, for example, in methods utilizing cytoplasmic male sterility, nuclear genetic male sterility, chemicals, genetic modification, genome editing, or a combination thereof.

Haploid induction systems have been developed for various plants to produce haploid tissues, plants and seeds. The haploid induction system can produce haploid plants from any genotype by crossing a selected line (as female) with an inducer line. Methods for obtaining haploid plants have also been disclosed in the art.

Thus, an aspect of this disclosure is a process for making a substantially homozygous PMWH149312414 progeny plant by producing or obtaining a seed from the cross of PMWH149312414 and another wheat plant and applying doubled haploid methods to the F₁ seed or F₁ plant, or to any successive filial generation. Such methods decrease the number of generations required to produce a variety with similar genetics or characteristics to PMWH149312414.

In one aspect, a process of making seed retaining the molecular marker profile of wheat variety PMWH149312414 is contemplated, such process comprising obtaining or producing F₁ seed for which wheat variety PMWH149312414 is a parent, inducing doubled haploids to create progeny without the occurrence of meiotic segregation, obtaining the molecular marker profile of wheat variety PMWH149312414, and selecting progeny that retain the molecular marker profile of wheat variety PMWH149312414.

Wheat variety PMWH149312414 can be crossed with one or more parental lines, followed by repeated selfing and selection, producing many new genetic combinations. Selected germplasm can be grown under unique and different geographical, climatic and soil conditions with further selections being made during and at the end of the growing season.

Wheat varieties that are highly homogeneous, homozygous and reproducible are useful as commercial varieties. There are many analytical methods, such as those described herein, which can be used to determine the homozygotic stability, phenotypic stability, and identity of these varieties produced or derived from variety PMWH149312414. Gel electrophoresis is particularly useful in wheat. Wheat variety identification can occur, for example, through electrophoresis of gliadin, glutenin, albumin and globulin, and total protein extracts.

Disclosed are plant breeding methods in which plant populations as well as individual plants are evaluated for general health, agronomics, and stability at one or more stages. These evaluations can include, but are not limited to, one or more of the following characteristics: plant architecture traits such as seedling coleoptile length, coleoptile color (presence of anthocyanin), juvenile plant growth habit, tillering, plant height, straw strength or lodging, flag leaf carriage at boot stage, leaf width and length, glaucosity of stems, leaves and spikes, pubescence of leaves and spikes, spike shape, spike density, spike awnedness, and plant color through-out stages of growth; plant growth characteristics, such as vernalization requirement, date for first stem joint emergence, heading date, flowering date, physiological maturity date and harvest maturity; tolerance to weather conditions, such as cold tolerance, resistance to heaving, tolerance to wet soils and standing water, drought and heat tolerance; and grain characteristics, such as grain yield, test weight, 1000 kernel weight, grain moisture, grain color, grain shape, grain protein, flour milling yield and baking characteristics.

During its development, wheat variety PMWH149312414 was assayed and/or planted in field trials and evaluated for a variety of traits and/or characteristics as compared to check varieties. The property(ies) of appropriate check varieties include but are not limited to varieties with a similar relative maturity, varieties known to be susceptible to one or more particular diseases, insect, pathogen, field condition, weather condition, soil type or condition, and/or crop management practice, varieties known to be tolerant or resistant to one or more particular diseases, insect, pathogen, field condition, weather condition, soil type or condition, and/or crop management practice, varieties comprising one or more particular marker locus, and/or varieties derived from another appropriate variety or having a particular pedigree. Appropriate choice of check varieties for comparison assures an appropriate baseline and valid qualitative or quantitative assessment of any test varieties.

In the development of PMWH149312414, the plants can be tested for various traits including, but not limited to grain yield, yield stability, test weight, heading date, harvest maturity, plant height, straw strength, pre-harvest sprout tolerance, lodging resistance, (early or late) maturity, resistance levels to leaf rust (brown rust), stem rust (black rust), stripe rust (yellow rust), tan spot, Septoria blotch (e.g., Septoria tritici, Septoria nodorum or Septoria avenae) blotch, Stagnospora nodorum blotch, spot blotch, powdery mildew, ergot (Claviceps purpurea), Fusarium (scab, Fusarium head blight), bacterial leaf streak, common bunt, wheat yellow mosaic virus, wheat streak mosaic virus and soilborne mosaic virus, barley yellow dwarf virus, orange blossom midge, Hessian Fly, wheat stem sawfly and grain characteristics such as flour yield, flour protein, and milling and baking characteristics.

Wheat variety PMWH149312414, being substantially homozygous, can be reproduced by planting seeds of the line, growing the resulting wheat plants under self-pollinating or sib-pollinating conditions, and harvesting the resulting seed, using techniques familiar to plant breeders.

In one aspect, wheat plants, plant parts and seeds are provided which have essentially all of the characteristics set forth in Table 1. In one aspect, wheat plants, plant parts and seeds are provided which have essentially all of the physiological and morphological characteristics of wheat variety PMWH149312414, or essentially all of the phenotypic characteristics of wheat variety PMWH149312414, representative seed having been deposited as disclosed herein.

Wheat variety PMWH149312414 can be further reproduced by tissue culture and regeneration. Tissue culture of various tissues of wheat and regeneration of plants therefrom is well known and widely published. Thus, in another aspect provided are cells which upon growth and differentiation produce wheat plants capable of having the physiological and morphological characteristics of wheat variety PMWH149312414.

As used herein, the term “plant parts” includes, without limitation, plant protoplasts, plant cell tissue cultures from which wheat plants can be regenerated, plant calli, plant clumps, plant cells, embryos, pollen, ovules, pericarp, seed, flowers, florets, heads, spikes, stems, stalks, leaves, roots, root tips, anthers, and the like. When indicating that a plant is crossed or selfed this indicates that any plant part of the plant can be used. For instance, the plant part does not need to be attached to the plant during the crossing or selfing, only the pollen might be used.

In one aspect, a wheat plant containing a locus conversion or an essentially derived variety of PMWH149312414 is provided. Essentially derived varieties may be obtained, for example, by the selection of a natural or induced mutant, or of a somaclonal variant, the selection of a variant individual from plants of the initial variety, backcrossing, or transformation by genetic engineering, or genome editing, from the repeated use of variety PMWH149312414 or being predominately derived from variety PMWH149312414.

A locus conversion refers to plants within a variety that have been modified in a manner that retains the overall genetics of the variety and further comprises one or more loci with a specific desired trait, such as male sterility, restoration of male sterility, insect, disease or herbicide resistance. Examples of single locus conversions include mutant genes, transgenes, gene-edited native genes and native traits finely mapped to a single locus. One or more locus conversion traits may be introduced into a single wheat variety. In one embodiment of the invention, the locus conversion is cytoplasmic or genic male sterility in the variety PMWH149312414, such as cytoplasmic male sterility (CMS) from Triticum timopheevii, or inactivated (mutated, deleted or gene-edited) wheat genes required for pollen production like MS1, MS5, MS9, MS22, MS26, or MS45, or the locus conversion is the introduction of one or more genes restoring such male sterility (like Rf genes restoring CMS or a functional native MS1, MS5, MS9, MS22, MS26, or MS45 wheat gene).

Transgenes and transformation methods provide means to engineer the genome of plants to contain and express heterologous genetic elements, including but not limited to foreign genetic elements, additional copies of endogenous elements, and/or modified versions of native or endogenous genetic elements, in order to alter at least one trait of a plant in a specific manner. Any heterologous DNA sequence(s), whether from a different species or from the same species, which are inserted into the genome using transformation, backcrossing, or other methods known to one of skill in the art are referred to herein collectively as transgenes. The sequences are heterologous based on sequence source, location of integration, operably linked elements, or any combination thereof. One or more transgenes of interest can be introduced into wheat variety PMWH149312414.

In some examples, transgenic variants of wheat variety PMWH149312414 are produced by introducing at least one transgene of interest into wheat variety PMWH149312414 by transforming wheat variety PMWH149312414 with a polynucleotide comprising the transgene of interest. In other examples, transgenic variants of wheat variety PMWH149312414 are produced by introducing at least one transgene by introgressing the transgene into wheat variety PMWH149312414 by crossing.

A transgenic variant of PMWH149312414 may contain at least one transgene but could contain at least 1, 2, 3, 4, 5, 6, 7, 8, 9, 10, or more transgenes. In another aspect, a transgenic variant of PMWH149312414 may contain no more than 15, 14, 13, 12, 11, 10, 9, 8, 7, 6, 5, 4, 3, or 2 transgenes.

In one example, a process for modifying wheat variety PMWH149312414 with the addition of a desired trait, said process comprising transforming a wheat plant of wheat variety PMWH149312414 with a transgene that confers a desired trait is provided. In other examples, the genome of wheat variety PMWH149312414 is modified by genome modification using techniques described herein, such as the CRISPR/Cas system adapted for use in plants. Therefore, transgenic wheat variety PMWH149312414 cells, plants, plant parts, and seeds produced from this process are provided.

In some aspects, one or more desired traits may include traits such as herbicide tolerance or resistance, insect tolerance or resistance, disease tolerance or resistance, resistance for bacterial, viral, or fungal disease, male fertility, male sterility, decreased phytate, modified fatty acid profile, modified fatty acid content, carbohydrate metabolism, protein content, or oil content. On other aspects, the desired trait is introduce via a transgene and the transgene is any known in the art or listed herein, including, but not limited to a polynucleotide conferring resistance to imidazolinone, dicamba, sulfonylurea, glyphosate, glufosinate, triazine, benzonitrile, cyclohexanedione, phenoxy propionic acid, and L-phosphinothricin; a polynucleotide encoding a Bacillus thuringiensis polypeptide, a polynucleotide encoding phytase, FAD-2, FAD-3, galactinol synthase or a raffinose synthetic enzyme, a polynucleotide providing resistance to Fusarium, Septoria, or various fungi, viruses or bacteria.

Numerous methods for plant transformation are known in the art, including biological, such as the use of Agrobacteria, and physical, such as biolistic and particle bombardment, plant transformation protocols. In addition, expression vectors and in vitro culture methods for plant cell or tissue transformation and regeneration of plants such as those known in the art can be used. Methods for producing transgenic plants have been developed and are well known in the art. As part of the disclosure, one of ordinary skill in the art may utilize any method of producing transgenic plants which is currently known or yet to be developed.

In general, methods to transform, modify, edit or alter plant endogenous genomic DNA include altering the plant native DNA sequence or a pre-existing transgenic sequence including regulatory elements, coding and non-coding sequences. These methods can be used, for example, to target nucleic acids to pre-engineered target recognition sequences in the genome. Such pre-engineered target sequences may be introduced by genome editing or modification, including methods like non-homologous end joining, homology-directed repair, base editing, and prime editing (e.g., Zong et al. (An engineered prime editor with enhanced editing efficiency in plants) in Nature Biotechnol., https://doi.org/10.1038/s41587-022-01254-w (2022)), with or without (use of ribonucleoprotein or RMPs) non-wheat DNA being inserted in the plant. As an example, a genetically modified plant variety can be generated using “custom” or engineered endonucleases such as meganucleases produced to modify plant genomes (see e.g., WO 2009/114321; Gao et al. (2010) Plant Journal 1:176-187). Another site-directed engineering method is through the use of zinc finger domain recognition coupled with the restriction properties of restriction enzyme. See e.g., Urnov, et al., (2010) Nat Rev Genet. 11 (9): 636-46; Shukla, et al., (2009) Nature 459 (7245): 437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39 (12) and Boch et al., (2009), Science 326 (5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9:39; The Cas/guide RNA-based system allows targeted cleavage of genomic DNA guided by a customizable small noncoding RNA in plants (see e.g., WO 2015026883A1, incorporated herein by reference). Several Cas genes are known and can be used, such as Cas9, Csn2, Cas4, Cas12a, Cas12b, Cas12c, Cas12d, Cas12e, Cas12f, Cas12g, Cas12h, Cas12i, Cas12k, C2c4, C2c8, and C2c9.

Plant transformation methods may involve the construction of an expression vector. Such a vector or recombinant construct comprises a DNA sequence that contains a coding sequence, such as a protein and/or RNA coding sequence under the control of or operatively linked to a regulatory element, for example a promoter. The vector or construct may contain one or more coding sequences and one or more regulatory elements.

A genetic trait which has been engineered into the genome of a particular wheat plant may then be moved into the genome of another variety using traditional breeding techniques that are well known in the plant breeding arts. For example, a backcrossing approach is commonly used to move a transgene or several transgenes (such as stacked transgenes located at one locus) from a transformed wheat variety into an elite wheat variety, and the resulting backcross conversion plant would then contain the transgene(s).

Various genetic elements can be introduced into the plant genome using transformation. These elements include, but are not limited to genes; coding sequences; inducible, constitutive, and tissue specific promoters; enhancing sequences; and signal and targeting sequences.

Provided are plants genetically engineered or transformed to express various phenotypes of agronomic interest. Expression of genes can be altered to enhance disease resistance, insect resistance, herbicide resistance, agronomic, grain quality, and other traits relative to a comparable wheat plant that does not contain the transformed element or to a comparable non-transformed plant. Transformation can also be used to insert DNA sequences which control or help control male-sterility. DNA sequences native to wheat as well as non-native DNA sequences can be transformed into the wheat plants described herein and used to alter levels of native or non-native proteins. Various promoters, targeting sequences, enhancing sequences, and other DNA sequences can be inserted into the genome for the purpose of altering the expression of proteins. Reduction or increase in the activity of specific genes by genetic transformation or modification can effect gene silencing, gene suppression or gene over expression in the plants described herein.

Many techniques for gene silencing are well known to one of skill in the art, including but not limited to, knock-outs, such as by insertion of a transposable element, antisense technology, (see U.S. Pat. Nos. 5,107,065; 5,453,566; and 5,759,829), co-suppression, CRISPS/Cas, RNA interference, virus-induced gene silencing, hairpin structures, ribozymes, oligonucleotide-mediated targeted modification (see, e.g., WO03/076574 and WO99/25853), Zn-finger targeted molecules (see, e.g., WO01/52620; WO03/048345; and WO00/42219), use of exogenously applied RNA (see, e.g., US20110296556), and other methods known to those of skill in the art or combinations of the above methods.

A genetic trait, engineered into a wheat plant using transformation, mutation or genome editing techniques can be transferred into another line using traditional breeding techniques that are well known in the plant breeding arts. The wheat plants described herein can be the donor or the recipient of the genetic trait. For example, a backcrossing approach can be used to move a transgene or mutated or edited native gene to an elite wheat variety to provide resulting progeny comprising a transgene, mutated or edited gene. As used herein, “crossing” can refer to a simple X by Y cross, or the process of backcrossing, depending on the context. The term “breeding cross” excludes the processes of selfing or sibbing.

Transgenic, mutated or gene-edited modified wheat plants described herein can be harvested to produce a foreign or modified protein in commercial quantities. The foreign or modified protein can be extracted from a tissue of interest, such as a seed, or from total biomass by known methods. The approximate chromosomal location of the integrated or modified DNA molecule can be determined from a genetic map generated, for example, via conventional RFLP, PCR, and SSR analysis.

Particular markers used for these purposes may include any type of marker and marker profile which provides a means of distinguishing varieties. A genetic marker profile can be used, for example, to identify plants of the same variety or related varieties or to determine or validate a pedigree. In addition to being used for identification of wheat variety PMWH149312414 and its plant parts, the genetic marker profile is also useful in developing a locus conversion of variety PMWH149312414.

Methods of isolating nucleic acids from wheat plants and methods for performing genetic marker profiles using SNP and SSR polymorphisms are well known in the art. SNPs are genetic markers based on a polymorphism in a single nucleotide. A marker system based on SNPs can be highly informative in linkage analysis relative to other marker systems in that multiple alleles may be present. Methods for analyzing polynucleotides from plants, plant parts or seeds described herein may include contacting a polynucleotide from the plant, plant part or seed, such as from wheat variety PMWH149312414 with a molecular marker or with modified nucleotides that facilitate sequencing of the polynucleotide. The polynucleotide may be isolated, separated or otherwise obtained from the plant, plant part or seed. Modified nucleotides such as dNTPs may be incorporated with the polynucleotides along with appropriate primers in a reaction mixture that facilitates sequencing. Sequencing can be done using any method known in the art.

SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by the polymerase chain reaction (PCR), thereby eliminating the need for labor-intensive Southern hybridization. PCR detection uses two oligonucleotide primers flanking the polymorphic segment of repetitive DNA. Repeated cycles of heat denaturation of the DNA, followed by annealing of the primers to their complementary sequences at low temperatures, and extension of the annealed primers with DNA polymerase, comprise a major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing varieties, all SSR profiles may be performed in the same lab.

The SSR profile of wheat plant PMWH149312414 can be used to identify plants comprising PMWH149312414 as a parent, since such plants will comprise the same homozygous alleles as PMWH149312414. Because the wheat variety is essentially homozygous at all relevant loci, most loci should have only one type of allele present. In contrast, a genetic marker profile of an F₁ progeny should be the sum of those parents, e.g., if one parent was homozygous for allele x at a particular locus, and the other parent homozygous for allele y at that locus, then the F₁ progeny will be xy (heterozygous) at that locus. Subsequent generations of progeny produced by selection and breeding are expected to be of genotype x (homozygous), y (homozygous), or xy (heterozygous) for that locus position. When the F₁ plant is selfed or sibbed for successive filial generations, the locus should be either x or y for that position.

In addition, plants and plant parts substantially benefiting from the use of PMWH149312414 in their development, such as PMWH149312414 comprising a backcross conversion, transgene, or genetic sterility factor, may be identified by having a molecular marker profile with a high percent identity to PMWH149312414. In an aspect, such a percent identity might be 95%, 96%, 97%, 98%, 99%, 99.5% or 99.9% identical to PMWH149312414.

The SSR profile of PMWH149312414 also can be used to identify essentially derived varieties and other progeny varieties developed from the use of PMWH149312414, as well as cells and other plant parts thereof. Progeny plants and plant parts produced using PMWH149312414 may be identified by having a molecular marker profile of at least 25%, 30%, 35%, 40%, 45%, 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% genetic contribution from PMWH149312414, as measured by either percent identity or percent similarity. Such progeny may be further characterized as being within a pedigree distance of PMWH149312414, such as within 1, 2, 3, 4 or 5 or fewer cross-pollinations to a wheat plant other than PMWH149312414 or a plant that has PMWH149312414 as a progenitor. Unique molecular profiles may be identified with other molecular tools such as SNPs and RFLPs.

While determining the SSR genetic marker profile of a plant as described above, several unique SSR profiles may also be identified that did not appear in either parent plant. Such unique SSR profiles may arise during the breeding process from recombination or mutation. A combination of several unique alleles provides a means of identifying a plant variety, an F₁ progeny produced from such variety, and further progeny produced from such variety.

A method comprising isolating nucleic acids, such as DNA, from a plant, a plant part, plant cell or a seed of the wheat varieties disclosed herein is provided. The method can include mechanical, electrical and/or chemical disruption of the plant, plant part, plant cell or seed, contacting the disrupted plant, plant part, plant cell or seed with a buffer or solvent, to produce a solution or suspension comprising nucleic acids, optionally contacting the nucleic acids with a precipitation agent to precipitate the nucleic acids, optionally extracting the nucleic acids, and optionally separating the nucleic acids such as by centrifugation or by binding to beads or a column, with subsequent elution, or a combination thereof. If DNA is being isolated, an RNase can be included in one or more of the method steps. The nucleic acids isolated can comprise all or substantially all of the genomic DNA sequence, all or substantially all of the chromosomal DNA sequence or all or substantially all of the coding sequences (cDNA) of the plant, plant part, or plant cell from which they were isolated. The nucleic acids isolated can comprise all, substantially all, or essentially all of the genetic complement of the plant. The nucleic acids isolated can comprise a genetic complement of the wheat variety. The amount and type of nucleic acids isolated may be sufficient to permit whole genome sequencing of the plant from which they were isolated or chromosomal marker analysis of the plant from which they were isolated.

The methods can be used to produce nucleic acids from the plant, plant part, seed or cell, which nucleic acids can be, for example, analyzed to produce data. The data can be recorded. The nucleic acids from the disrupted cell, the disrupted plant, plant part, plant cell or seed or the nucleic acids following isolation or separation can be contacted with primers and nucleotide bases, and/or a polymerase to facilitate PCR sequencing or marker analysis of the nucleic acids. In some examples, the nucleic acids produced can be sequenced or contacted with markers to produce a genetic profile, a molecular profile, a marker profile, a haplotype, or any combination thereof. In some examples, the genetic profile or nucleotide sequence is recorded on a computer readable medium. In other examples, the methods may further comprise using the nucleic acids produced from plants, plant parts, plant cells or seeds in a plant breeding program, for example in making crosses, selection and/or advancement decisions in a breeding program. Crossing includes any type of plant breeding crossing method, including but not limited to crosses to produce hybrids, outcrossing, selfing, backcrossing, locus conversion, introgression and the like.

Favorable genotypes and or marker profiles, optionally associated with a trait of interest, may be identified by one or more methodologies. In some examples one or more markers are used, including but not limited to AFLPs, RFLPs, ASH, SSRs, SNPs, indels, padlock probes, molecular inversion probes, microarrays, sequencing, and the like. In some methods, a target nucleic acid is amplified prior to hybridization with a probe. In other cases, the target nucleic acid is not amplified prior to hybridization, such as methods using molecular inversion probes (see, for example Hardenbol et al. (2003) Nat Biotech 21:673-678). In some examples, the genotype related to a specific trait is monitored, while in other examples, a genome-wide evaluation including but not limited to one or more of marker panels, library screens, association studies, microarrays, gene chips, expression studies, or sequencing such as whole-genome resequencing and genotyping-by-sequencing (GBS) may be used. In some examples, no target-specific probe is needed, for example by using sequencing technologies, including but not limited to next-generation sequencing methods (see, for example, Metzker (2010) Nat Rev Genet 11:31-46; and, Egan et al. (2012) Am J Bot 99:175-185) such as sequencing by synthesis (e.g., Roche 454 pyrosequencing, Illumina Genome Analyzer, and Ion Torrent PGM or Proton systems), sequencing by ligation (e.g., SOLID from Applied Biosystems, and Polnator system from Azco Biotech), and single molecule sequencing (SMS or third-generation sequencing) which eliminate template amplification (e.g., Helicos system, and PacBio RS system from Pacific BioSciences). Further technologies include optical sequencing systems (e.g., Starlight from Life Technologies), and nanopore sequencing (e.g., GridION from Oxford Nanopore Technologies). Each of these may be coupled with one or more enrichment strategies for organellar or nuclear genomes in order to reduce the complexity of the genome under investigation via PCR, hybridization, restriction enzyme (see, e.g., Elshire et al. (2011) PLOS ONE 6: e19379), and expression methods. In some examples, no reference genome sequence is needed in order to complete the analysis. Variety PMWH149312414 and its plant parts can be identified through a molecular marker profile. Such plant parts may be either diploid or haploid.

As described herein, genes or coding sequences can be expressed in transformed plants. More particularly, plants can be genetically engineered to express various phenotypes of agronomic interest. A single gene or locus conversion or at least about 1, 2, 3, 4, 5, 6, 7, 8, 9, 10, 11, 12, 13, 14, 15, 16, 17, 18, 19, 20, 25, 30, 35 or 40 or more genes or locus conversions and less than about 100, 90, 80, 70, 60, 50, 40, 30, 20, 15, or 10 genes or locus conversions may be introduced into a plant or comprised in the genome of the wheat plant. Combinations or stacks of two or more genes or coding sequences described herein can be used. Through the transformation, mutation or genome modification of wheat the expression of genes can be modulated to enhance disease resistance, insect resistance, herbicide resistance, water stress tolerance and agronomic traits as well as grain quality traits. Transformation, mutation or genome editing can also be used to insert or modify (including inactivate) DNA sequences which control or alter male-sterility. DNA sequences native to wheat can be modified as well as native and non-native DNA sequences can be introduced into wheat and used to modulate levels of native or non-native proteins. The sequences introduced can be native or modified wheat sequences, or can be heterologous comprising a coding sequence operably linked to a heterologous regulatory element, such as a promoter.

Exemplary genes which can be targeted include, but are not limited to, genes that confer resistance to pests such as Hessian fly, wheat stem sawfly, cereal leaf beetle, and/or green bug or disease, to pathogens Cladosporium fulvum, Pseudomonas syringae, Fusarium graminearum Schwabe, wheat rusts, Septoria tritici, Septoria nodorum, powdery mildew, Helminthosporium diseases, smuts, bunts, Fusarium diseases, bacterial diseases, and viral diseases.

Other genes, coding sequences or targets which can be used include those encoding Bacillus thuringiensis protein, a derivative thereof or a synthetic polypeptide modeled thereon. Examples of Bacillus thuringiensis transgenes encoding an endotoxin and being genetically engineered are given in the following patents and patent applications and hereby are incorporated by reference for this purpose: U.S. Pat. Nos. 5,188,960; 5,689,052; 5,880,275; 8,809,654; WO 91/14778; WO 99/31248; WO 01/12731; WO 99/24581; WO 97/40162 and U.S. application Ser. Nos. 10/032,717; 10/414,637; and Ser. No. 10/606,320.

Other genes, coding sequences or targets which can be used include those encoding an insect-specific hormone or pheromone such as an ecdysteroid and juvenile hormone, a variant thereof, a mimetic based thereon, or an antagonist or agonist thereof, an insect diuretic hormone receptor, such as an allostatin (see also U.S. Pat. No. 5,266,317 incorporated herein by reference for this purpose), an enzyme responsible for a hyper accumulation of a monoterpene, a sesquiterpene, a steroid, hydroxamic acid, a phenylpropanoid derivative or another non-protein molecule with insecticidal activity, an enzyme involved in the modification, including the post-translational modification, of a biologically active molecule, for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic; a molecule that stimulates signal transduction, for example mung bean calmodulin cDNA clones and maize calmodulin cDNA clones; a hydrophobic peptide (see U.S. Pat. Nos. 5,580,852 and 5,607,914 incorporated herein by reference for this purpose); a membrane permease, a channel former or a channel blocker, for example, Cecropin-beta lytic peptide analog conferring Pseudomonas solanacearum resistance; an insect-specific antibody or an immunotoxin derived therefrom, or a virus-specific antibody; a developmental-arrestive protein such as a endopolygalacturonase-inhibiting protein or a ribosome-inactivating gene; genes involved in the Systemic Acquired Resistance (SAR) Response and/or the pathogenesis related genes,

In some aspects, coat protein-mediated resistance can be conferred in plants against one or more of wheat virus(es). Such resistance may be conferred using, for example, a viral-invasive protein or a complex toxin derived therefrom.

In some aspects, genes, coding sequences or targets which can be used include, without limitation, antifungal genes (see, for example, US Publication No: 20020166141 incorporated herein by reference for this purpose); detoxification genes, such as for fumonisin, beauvericin, moniliformin and zearalenone and their structurally related derivatives (see, for example, U.S. Pat. No. 5,792,931 incorporated herein by reference for this purpose); cystatin and cysteine proteinase inhibitors (see for example, U.S. Patent Publication Serial No: 20050102717 incorporated herein by reference for this purpose), defensin genes (see for example, PCT Public WO03000863 and U.S. Patent Publication Serial No: 20030041348); and genes conferring resistance to nematodes, see for example, WO 03/033651.

Genes, coding sequences, or targets that confer resistance to a herbicide are described, for example, in U.S. Pat. No. 8,809,654, which is incorporated by reference herein for this purpose. Examples include genes or coding sequences encoding acetohydroxy acid synthase, a chimeric protein of rat cytochrome P4507A1, yeast NADPH-cytochrome P450 oxidoreductase, glutathione reductase, superoxide dismutase, phosphotransferases, ALS and AHAS enzymes and other genes or coding sequences which confer resistance to a herbicide such as an imidazalinone or a sulfonylurea (see also, U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, each of which are incorporated herein by reference for this purpose); Glyphosate or glufosinate resistance can also be conferred using, for example, sequences encoding mutant 5-enolpyruvl-3-phosphikimate synthase (EPSP), aroA genes, phosphinothricin acetyl transferase (PAT), glyphosate oxidoreductase enzyme, glyphosate N-acetyltransferase, glutamine synthetase, Streptomyces hygroscopicus phosphinothricin acetyl transferase (bar) genes), and pyridinoxy or phenoxy proprionic acids and cycloshexones (ACCase inhibitor-encoding genes).

Triazine resistance can be conferred using, for example, psbA and gs+ genes, sequences encoding a benzonitrile (nitrilase gene) such as disclosed in U.S. Pat. No. 4,810,648 incorporated herein by reference for this purpose.

Resistance to herbicides which target Protoporphyrinogen oxidase (protox) can also be conferred such resistance being described in U.S. Pat. Nos. 6,288,306, 6,282,837, 5,767,373 and international publication WO 01/12825 the disclosures of each of which are herein incorporated by reference for this purpose.

Genes, coding sequences, or targets that confer or improve grain quality include, without limitation, altered fatty acids (for example, oleic, linoleic, linolenic), altered phosphorus content (for example, using phytase), altered carbohydrates such as modulating the branching pattern of starch or altering thioredoxin, Bacillus subtilis levansucrase gene, Bacillus licheniformis alpha-amylase, tomato invertase, alpha-amylase gene, starch branching enzyme II, UDP-D-xylose 4-epimerase, Fragile 1 and 2, Ref1, HCHL, C4H, high oil seed such as by modification of starch levels (AGP). Fatty acid modification genes mentioned above may also be used to affect starch content and/or composition through the interrelationship of the starch and oil pathways, altered content or composition of antioxidants such as tocopherol or tocotrienols, such as using a phytl prenyl transferase (ppt), or through alteration of a homogentisate geranyl gernayl transferase (hggt). Genes, coding sequences, or targets that can be targets to confer or improve grain quality are disclosed in, for example, see U.S. Pat. Nos. 8,809,654, 6,787,683, 6,531,648, 6,423,886, 6,232,529, 6,197,561, 6,825,397, U.S. Patent Publication Nos. 2003/0079247, US2003/0204870, US2004/0034886 international PCT publications WO 02/42424, WO 98/22604, WO 03/011015, WO02/057439, WO03/011015, WO 99/10498, WO 00/68393, and WO 03/082899.

Genes, coding sequences or targets for altered essential seed amino acids, such as one or more of lysine, methionine, threonine, tryptophan or altered sulfur amino acid content are also provided, and can be used in the methods and plants described herein.

Genes, coding sequences or targets that control or alter male sterility and methods for conferring male sterility and male sterile plants are provided. There are several methods of conferring genetic male sterility available, such as disclosed in U.S. Pat. Nos. 8,809,654, 4,654,465 and 4,727,219, 3,861,709, 3,710,511, 5,432,068, the disclosures of each of which are herein incorporated by reference for this purpose. For additional examples of nuclear male and female sterility systems and genes, see also, U.S. Pat. Nos. 5,859,341; 6,297,426; 5,478,369; 5,824,524; 5,850,014; and 6,265,640; each of which are hereby incorporated by reference for this purpose.

Genes, coding sequences or targets that create a site for site specific DNA integration can also be used such as the introduction of FRT sites that may be used in the FLP/FRT system and/or Lox sites that may be used in the Cre/Loxp system. Other systems that may be used include the Gin recombinase of phage Mu, the Pin recombinase of E. coli, and the R/RS system of the pSR1 plasmid.

Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress are provided. For example, see: U.S. Pat. Nos. 8,809,654, 5,892,009, 5,965,705, 5,929,305, 5,891,859, 6,417,428, 6,664,446, 6,706,866, 6,717,034, 6,801,104, 6,177,275, 6,107,547, 6,084,153, US Patent Publication Nos. 2004/0148654, 2004/0237147, 2003/0166197, 2004/0128719, 2004/0098764, 2004/0078852, international PCT application WO2000060089, WO2001026459, WO2001035725, WO 00/73475; WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521, WO01/36596 and WO9938977, WO2000/006341, WO04/090143, WO0202776, WO2003052063, WO0164898, and WO200032761, the disclosures of each of which are herein incorporated by reference in its entirety for this purpose.

Other genes and transcription factors that affect plant growth and agronomic traits such as yield, flowering, plant growth and/or plant structure, can be introduced or introgressed into plants, see e.g. WO97/49811 (LHY), WO98/56918 (ESD4), WO97/10339 and U.S. Pat. No. 6,573,430 (TFL), U.S. Pat. No. 6,713,663 (FT), WO96/14414 (CON), WO96/38560, WO01/21822 (VRN1), WO00/44918 (VRN2), WO99/49064 (GI), WO00/46358 (FRI), WO97/29123, U.S. Pat. Nos. 6,794,560, 6,307,126 (GAI), WO99/09174 (D8 and Rht), and WO2004076638 and WO2004031349 (transcription factors), the disclosures of each of which are herein incorporated by reference.

Genes that confer agronomic enhancements, nutritional enhancements, or industrial enhancements can also be used. Such genes are described for example in U.S. Pat. No. 8,809,654, the disclosure of which is herein incorporated by reference in for this purpose. Such enhancements include, without limitation, improved tolerance to water stress from drought or high saltwater condition. See, e.g., U.S. Pat. Nos. 5,981,842, 5,780,709, international patent applications WO 92/19731, WO 92/19731 the disclosures of each of which is herein incorporated by reference for this purpose.

In some aspects, methods of treating PMWH149312414 with a mutagen and the plant produced by mutagenesis of PMWH149312414 are provided. Backcross conversions of wheat variety PMWH149312414 are also described. A backcross conversion occurs when modified or non-native DNA sequences are introduced through traditional (non-transformation) breeding techniques, such as backcrossing. DNA sequences, whether naturally occurring, modified or transgenes, may be introduced using these traditional breeding techniques. Desired traits transferred through this process include, but are not limited to, nutritional enhancements, industrial enhancements, (fungal or viral) disease resistance, insect resistance, herbicide resistance, agronomic enhancements, grain quality enhancement, waxy starch, breeding enhancements, seed production enhancements, and male sterility. Descriptions of some of the cytoplasmic male sterility genes, nuclear male sterility genes, chemical hybridizing agents, male fertility restoration genes, and methods of using the aforementioned are discussed in “Hybrid Wheat” by K. A. Lucken (pp. 444-452 In Wheat and Wheat Improvement, ed. Heyne, 1987). Examples of genes for other traits which can be used with the methods, plants and plant parts described herein include: Leaf rust resistance genes (Lr series such as Lr1, Lr10, Lr21, Lr22, Lr22a, Lr32, Lr37, Lr41, Lr42, and Lr43), Fusarium head blight-resistance genes (QFhs.ndsu-3B and QFhs.ndsu-2A), Powdery Mildew resistance genes (Pm21), common bunt resistance genes (Bt-10), and wheat streak mosaic virus resistance gene (Wsm1), Russian wheat aphid resistance genes (Dn series such as Dn1, Dn2, Dn4, Dn5), (genic or CMS) male sterility genes, restorer genes for male sterility, Black stem rust resistance genes (Sr38), Yellow rust resistance genes (Yr series such as Yr1, YrSD, Yrsu, Yr17, Yr15, YrH52), aluminum tolerance genes (Alt(BH)), dwarf genes (Rht), vernalization genes (Vrn), Hessian fly resistance genes (H9, H10, H21, H29), grain color genes (R/r), glyphosate resistance genes (EPSPS), glufosinate genes (bar, pat) and water stress tolerance genes (Hval, mtID). The trait of interest is transferred from the donor parent to the recurrent parent, in this case, the wheat plant disclosed herein. Single gene traits, whether naturally occurring, induced by mutation or genetically altered, may result from either the transfer of a dominant allele or a recessive allele. Selection of progeny containing the trait of interest is done by direct selection for a trait associated with a dominant allele. Selection of progeny for a trait that is transferred via a recessive allele requires growing and selfing the first backcross to determine which plants carry the recessive alleles. Recessive traits may require additional progeny testing in successive backcross generations to determine the presence of the gene of interest.

Methods of developing a backcross conversion PMWH149312414 wheat plant are provided including the step of repeated backcrossing to wheat variety PMWH149312414. The number of backcrosses made may be 2, 3, 4, 5, 6, 7, 8 or greater, and fewer than 50, 40, 30, 25, 20, 15, 10, 9, or 8. The specific number of backcrosses used will depend upon the genetics of the donor parent and whether molecular markers are utilized in the backcrossing program. Provided are plants and plant populations that are produced from backcrossing methods, transformation, locus conversion, or otherwise produced, and combinations thereof and that retain at least 70%, 75%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, or 99.9% or 99.95%, 99.98%, 99.985%, 99.99% or 99.995% of the genetic profile of wheat variety PMWH149312414. The percentage of the genetics retained in the backcross conversion may be measured by either pedigree analysis or through the use of genetic techniques such as molecular markers or electrophoresis. Such methods and techniques are described in U.S. Pat. No. 8,809,654, the disclosure of which is herein incorporated by reference for this purpose. The backcross conversion or locus conversion developed from this method may be similar to PMWH149312414 for the results listed in Tables 1, 2A and 2B. Such similarity may be measured by a side-by-side phenotypic comparison, with differences and similarities determined at a 5% significance level, when appropriate in environmental conditions that account for the trait being transferred. For example, herbicide should not be applied in the phenotypic comparison of herbicide resistant backcross conversion of PMWH149312414 when compared back to PMWH149312414.

Described are methods for using wheat variety PMWH149312414 in plant breeding and plants and plant populations produced by such methods. For example, wheat variety PMWH149312414 can be crossed with another variety of wheat to form a first generation population of F₁ plants. This first generation population of F₁ plants will comprise an essentially complete set of the alleles of wheat variety PMWH149312414. Also provided are methods and plants which use transgenic or backcross conversions of wheat variety PMWH149312414 to produce first generation F₁ plants.

A method of developing a PMWH149312414-progeny wheat plant comprising crossing PMWH149312414 with a second wheat plant and performing a breeding method is also described. An exemplary method for producing a line derived from wheat variety PMWH149312414 is as follows. Wheat variety PMWH149312414 is crossed with another variety of wheat, such as an elite variety. The F₁ seed derived from this cross is grown to form a homogeneous population. The F₁ seed contains one set of the alleles from variety PMWH149312414 and one set of the alleles from the other wheat variety. The F₁ genome is 50% variety PMWH149312414 and 50% of the other elite variety. The F₁ seed is grown and allowed to self, thereby forming F₂ seed. On average the F₂ seed would have derived 50% of its alleles from variety PMWH149312414 and 50% from the other wheat variety, but various individual plants from the population can have a much greater percentage of their alleles derived from PMWH149312414. The F₂ seed is grown and selection of plants made based on visual observation and/or measurement of traits. The PMWH149312414-derived progeny that exhibit one or more of the desired PMWH149312414-derived traits are selected and each plant is harvested separately. This F₃ seed from each plant is grown in individual rows and allowed to self. Then selected rows or plants from the rows are harvested and threshed individually. The selections based on visual observation and/or measurements for desirable traits of the plants, such as one or more of the desirable PMWH149312414-derived traits are made. The process of growing and selection is repeated any number of times until a homozygous PMWH149312414-derived wheat plant is obtained. The homozygous PMWH149312414-derived wheat plant contains desirable traits derived from wheat variety PMWH149312414, some of which may not have been expressed by the other original wheat variety to which wheat variety PMWH149312414 was crossed and some of which may have been expressed by both wheat varieties but now would be at a level equal to or greater than the level expressed in wheat variety PMWH149312414. The homozygous PMWH149312414-derived wheat plants have, on average, 50% of their genes derived from wheat variety PMWH149312414, but various individual plants from the population would have a much greater percentage of their alleles derived from PMWH149312414. The breeding process, of crossing, selfing, and selection may be repeated to produce another population of PMWH149312414-derived wheat plants with, on average, 25% of their genes derived from wheat variety PMWH149312414, and with various individual plants from the population having a much greater percentage of their alleles derived from PMWH149312414. Homozygous PMWH149312414-derived wheat plants that have received PMWH149312414-derived traits are also provided.

In some instances, selection may or may not occur at every selfing generation, selection may occur before or after the actual self-pollination process occurs, or individual selections may be made by harvesting individual spikes, plants, rows or plots at any point during the breeding process described herein. In addition, doubled haploid breeding methods may be used at any step in the process. In one aspect, the population of plants produced at each and any generation of selfing, each such population consisting of plants containing approximately 50% of its genes from wheat variety PMWH149312414, 25% of its genes from wheat variety PMWH149312414 in the second cycle of crossing, selfing, and selection, 12.5% of its genes from wheat variety PMWH149312414 in the third cycle of crossing, selfing, and selection, and so on.

Also disclosed are methods of obtaining a homozygous PMWH149312414-derived wheat plant by crossing wheat variety PMWH149312414 with another variety of wheat and applying doubled haploid methods to the F₁ seed or F₁ plant or to any generation of PMWH149312414-derived wheat obtained by the selfing of this cross.

Still further, methods for producing PMWH149312414-derived wheat plants are provided by crossing wheat variety PMWH149312414 with a wheat plant and growing the progeny seed, and repeating the crossing or selfing along with the growing steps with the PMWH149312414-derived wheat plant from 1 to 2 times, 1 to 3 times, 1 to 4 times, or 1 to 5 times. Thus, any and all methods using wheat variety PMWH149312414 in breeding, including selfing, pedigree breeding, backcrossing, hybrid production and crosses to populations are provided. Unique starch profiles, molecular marker profiles and/or breeding records can be used to identify the progeny lines or populations derived from these breeding methods.

Also disclosed are methods of harvesting the grain of variety wheat variety PMWH149312414 and using the grain as seed for planting. Aspects include cleaning the seed, treating the seed, and/or conditioning the seed. Cleaning the seed includes removing foreign debris such as weed seed and removing chaff, plant matter, from the seed. Conditioning the seed can include controlling the temperature and rate of dry down and storing seed in a controlled temperature environment. Seed treatment is the application of a composition to the seed such as a coating or powder. Seed material can be treated, typically surface treated, with a composition comprising combinations of chemical or biological herbicides, herbicide safeners, pesticides, insecticides, fungicides, nutrients, germination inhibitors, germination promoters, cytokinins, nutrients, plant growth regulators, antimicrobials, and activators, bactericides, nematicides, avicides, or molluscicides. These compounds are typically formulated together with further carriers, surfactants or application-promoting adjuvants customarily employed in the art of formulation. The coatings may be applied by impregnating propagation material with a liquid formulation or by coating with a combined wet or dry formulation. Examples of the various types of compounds that may be used as seed treatments are provided in The Pesticide Manual: A World Compendium, C. D. S. Tomlin Ed., published by the British Crop Production Council. Some specific seed treatments that may be used on crop seed include, but are not limited to, abscisic acid, acibenzolar-S-methyl, avermectin, amitrol, azaconazole, azospirillum, azoxystrobin, bacillus, Bacillus subtilis, Bacillus simplex, Bacillus firmus, Bacillus amyloliquefaciens, Pasteuria genus (e.g. P. nishizawae), bradyrhizobium, captan, carboxin, chitosan, clothianidin, copper, cyazypyr, difenoconazole, etidiazole, fipronil, fludioxonil, fluquinconazole, flurazole, fluxofenim, GB126, Harpin protein, imazalil, imidacloprid, isofavenoids, lipo-chitooligosaccharide, mancozeb, manganese, maneb, mefenoxam, metalaxyl, metconazole, PCNB, penflufen, penicillium, penthiopyrad, permethrine, picoxystrobin, prothioconazole, pyraclostrobin, rynaxypyr, S-metolachlor, saponin, sedaxane, TCMTB, tebuconazole, thiabendaxole, thiamethoxam, thiocarb, thiram, tolclofos-methyl, triadimenol, trichoderma, trifloxystrobin, triticonazole and/or zinc.

Seed varieties and seeds with specific genetic resistance traits can be tested to determine which seed treatment options and application rates will complement such varieties and genetic resistance traits in order to enhance yield. For example, a variety with good yield potential but loose smut susceptibility will benefit from the use of a seed treatment that provides protection against loose smut. Likewise, a variety encompassing a genetic resistance trait conferring insect resistance will benefit from the second mode of action conferred by the seed treatment. Further, the good root establishment and early emergence that results from the proper use of a seed treatment will result in more efficient nitrogen use, a better ability to withstand drought and an overall increase in yield potential of a variety or varieties containing a certain trait when combined with a seed treatment.

Wheat variety PMWH149312414 has traits and characteristics that distinguish it from other wheat varieties. Table 1 provides a description of the traits used to measure or characterize a wheat variety such as variety PMWH149312414 and the scoring ranges used for such traits. Tables 2A and 2B provide agronomic, grain quality and disease reaction characters of PMWH149312414 variety compared to relevant US and Canadian Spring Wheat check varieties.

TABLE 1
VARIETY DESCRIPTION INFORMATION

Kind: Common
Seasonal growth habit: Spring
Juvenile growth habit: Erect
Leaf color at boot: Blue-green
Flag leaf at boot: Re-curved, twisted, waxed
Auricle color: Purple
Days to 50% heading (average): 55
Anther color: Yellow
Anthocyanin: Absent
Plant height (average, in cms): 77
Internodes: Hollow
Spike shape: Elliptical
Spike density: Mid-dense
Spike curvature: Inclined
Awn type: Awned
Awn color: Tan
Glume pubescence: Present
Seed color: Red
Seed shape: Elliptical
Cheeks: Rounded
Brush size: Short

TABLE 2A
CHARACTERISTICS OF PMWH149312414 COMPARED
TO RELEVANT CHECK VARIETIES

| | | | | | | Restore Self | Restore Self |
|---|---|---|---|---|---|---|---|
| | | Grain | | | | Fertility at | Fertility at |
| | Yield | Protein | Ht | Days to | Weight | Maturity | Flowering |
| Material Name | MT/ha | % | (cm) | heading | Lbs/Bu | (1-9 Scale)* | (1-9 Scale)* |
| SY Valda | 4.76 | 13.7 | 75 | 48.7 | 75.1 | 9.0 | 9.0 |
| Faller | 4.67 | 13.9 | 82 | 49.1 | 74.4 | 9.0 | 9.0 |
| PMWH149312414 | 4.08 | 14.7 | 75 | 49.2 | 70.9 | 8.9 | 9.0 |

*Scale of 1-9 where 1 = poor/no restoration of fertility, 9 = complete or almost complete restoration of fertility

TABLE 2B
CHARACTERISTICS OF PMWH149312414 COMPARED
TO RELEVANT CHECK VARIETIES

| | | | Yellow | Leaf |
|---|---|---|---|---|
| | | FHB | Rust | Rust |
| | Lodging | Resistance | Resistance | Resistance |
| | (1-9 | (1-9 | (1-9 | (1-9 |
| Material Name | Scale)** | Scale)** | Scale)** | Scale)** |
| SY Valda | 3 | 4 | 5 | 3 |
| Faller | 4 | 4 | 7 | 4 |
| PMWH149312414 | 2 | 6 | 7 | 4 |

**Scale of 1-9 where 9 = excellent or resistant, 1 = poor or susceptible

### Deposit

A deposit of the BASF SE proprietary wheat variety PMWH149312414 disclosed above and recited in the appended claims is maintained by BASF SE. A deposit under the Budapest Treaty will be made with the Provasoli-Guillard National Center for Marine Algae and Microbiota, Bigelow Laboratory for Ocean Sciences (NCMA), 60 Bigelow Drive, East Boothbay, Maine 04544. Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period.

1. A plant, plant part, seed, or plant cell of wheat variety PMWH149312414, wherein a representative sample of seed of said variety is deposited under NCMA No 202408008.

2. A wheat seed produced from (i) selfing the plant or plant part of claim 1 or (ii) crossing of the plant or plant part of claim 1 with a different wheat plant or plant part.

3. A wheat plant or plant part produced by growing the wheat seed of claim 2.

4. The wheat seed of claim 2, wherein the seed is an F₁ hybrid wheat seed.

5. A method for producing a progeny seed, the method comprising crossing the wheat plant of claim 3, to a plant of wheat variety PMWH149312414, wherein a representative sample of seed of said variety has been deposited under NCMA No. 202408008, and producing a progeny seed.

6. The method of claim 5, wherein the method further comprises crossing a plant grown from the progeny seed to a plant of wheat variety PMWH149312414, and producing a backcrossed seed.

7. The backcrossed seed produced by the method of claim 6.

8. A method for producing a second wheat plant, the method comprising applying plant breeding techniques to the wheat plant or plant part of claim 3, wherein application of said techniques results in the production of a second wheat plant.

9. A method for producing a second wheat plant, the method comprising doubling haploid seed generated from a cross of the wheat plant or plant part of claim 3 with a different wheat plant.

10. A method comprising cleaning the seed of claim 4.

11. The seed of claim 4, further comprising a seed treatment on the surface of the seed.

12. A method for producing nucleic acids, the method comprising isolating nucleic acids from the plant, plant part, seed, or plant cell of claim 4.

13. A plant, plant part, seed, or plant cell of wheat variety PMWH149312414, wherein a representative sample of seed of said variety has been deposited under NCMA No. 202408008, further comprising a locus conversion.

14. The plant, plant part, seed, or plant cell of claim 13, wherein the locus conversion comprises a transgene.

15. The plant, plant part, seed, or plant cell of claim 13, wherein the locus conversion confers a trait selected from the group of male sterility, male fertility, abiotic stress tolerance, altered phosphate content, altered protein, altered antioxidants, altered fatty acids, altered essential amino acids, altered carbohydrates, herbicide resistance, insect resistance, and disease resistance, wherein the altered trait is compared with a similar plant not comprising the locus conversion.

16. A tissue culture produced from the plant, plant part, seed, or plant cell of claim 15.

17. A wheat seed produced by crossing the plant of claim 15 with a different wheat plant.

18. A wheat plant produced by growing the wheat seed of claim 17.

19. A method of producing a second wheat plant comprising applying plant breeding techniques to the plant of claim 18, wherein application of said techniques results in the production of a second wheat plant.
